(19) United States
(12) Patent Application Publication
(10) Pub. No.: US 2004/0014639 A1
(43) Pub. Date: Jan. 22, 2004

(54) Product and method for control of obesity

(76) Inventors: Christopher Ian Belyea, Victoria (AU); Mark Heffernan, Victoria (AU); Roger Summers, Victoria (AU)

Correspondence Address: KLARQUIST SPARKMAN, LLP, 121 SW SALMON STREET, SUITE 1600, PORTLAND, OR, 97204, US

(21) Appl. No.: 10/363,094

(22) PCT Filed: Aug. 29, 2001
(86) PCT No.: PCT/AU01/01086

(30) Foreign Application Priority Data
Sep. 1, 2000 (AU) PQ 9817

(51) Int. Cl. A61K 31/137; A61K 38/22

(52) U.S. Cl. 514/2; 514/653

(57) ABSTRACT

This invention relates to the prevention and treatment of obesity in mammals, especially humans. In particular, the invention relates to a method of treating obesity in a mammal, comprising the step of administering to the mammal a therapeutically-effective amount of an agent which increases the expression of beta-3 adrenergic receptors in the mammal, together with an agonist of the beta 3 adrenergic receptor.

### FIELD OF THE INVENTION

This invention relates to the prevention and treatment of obesity in mammals, especially humans. In particular, the invention relates to the use of two known obesity control agents in a synergistic relationship to enhance the control of obesity via the regulation of lipid and carbohydrate metabolism.

### BACKGROUND OF THE INVENTION

In Australian patent No. 693478 by Monash University, we described the use of a peptide derived from the carboxyl-terminal sequence of human growth hormone, or corresponding regions from growth hormone of other mammalian species, for the control of obesity. This fragment of growth hormone, like growth hormone itself, has the ability to modulate lipid metabolism. In particular, a synthetic peptide corresponding to amino acid residues 177-191 of the human growth hormone sequence (hereinafter referred to as hGH 177-191) was found to reduce body weight gain and adipose tissue mass in a model system for obesity, the C57Bl/6J (Ob/Ob) mouse. A subsequent application, PCT/AU98/00724 by Metabolic Pharmaceuticals Ltd, discloses analogues of the hGH177-191 peptide which share this activity. The entire disclosures of AU693478 and PCT/AU98/00724 are incorporated herein by this reference. All of the studies described in the two earlier specifications were carried out using administration of the peptide by injection, but these peptides have since been found to be orally available to a substantial extent, allowing practical oral administration.

Beta 3 agonists are another class of potential obesity drug which are currently in development by a number of pharmaceutical companies. Although rodents have large amounts of brown fat, which predominantly houses the beta 3 adrenergic receptors, humans have relatively little or no brown fat. Therefore the relative importance of the mediation of the lipolytic effects through the beta 3 adrenergic receptor is lower in the human than in the rodent. Nevertheless, the human response to beta 3 agonists is significant since the beta 3 adrenergic receptor is also present in the adipocytes of the white fat. It is expected that the newer class of beta 3 agonists, designed to optimally act on the human beta 3 adrenergic receptor, will produce a significant lipolytic effect in human subjects, and thereby provide an improved obesity treatment.

We have now found that at least one of the mechanisms of action of growth hormone and the growth hormone fragments is to increase the expression of beta-3 adrenergic receptors in rodent white and brown adipocytes, and thereby to potentiate the lipolytic effects of endogenous adrenaline or an exogenously administered agonist at the beta 3 adrenergic receptor. This finding means that a combination therapy using growth hormone, growth hormone fragments, or compounds acting by a similar mechanism, together with beta 3 agonists, will enhance the obesity-reducing properties of the beta 3 agonists.

### SUMMARY OF THE INVENTION

According to a first aspect, the invention provides a method of treating obesity in a mammal, comprising the step of administering to the mammal in thereof a therapeutically-effective amount of an agent which increases the expression of beta-3 adrenergic receptors in the mammal, together with an agonist of the beta 3 adrenergic receptor of the mammal.

Preferably, the agent which increases the expression of the beta 3 adrenergic receptors is a growth hormone, a lipid metabolic growth hormone fragment, or a non-peptide analogue thereof.

Preferably, the agonist of the beta 3 adrenergic receptor of the mammal is a selective beta 3 agonist to the human beta 3 receptor, and the mammal is a human.

Alternatively, the mammal may be a pig, and the beta 3 agonist may be either a selective or non-selective agonist to the beta 3 adrenergic receptor, such as ractopamine.

For the purposes of this specification, the term “lipid metabolic growth hormone fragment” or “growth hormone fragment” is to be understood to mean a polypeptide fragment from the carboxy-terminal region of the amino acid sequence of a mammalian growth hormone, which has one or more of the following biological activities:

(a) ability to reduce body weight gain and adipose tissue mass in a homologous mammal,

(b) ability to reduce lipogenic activity, and

(c) ability to stimulate lipolysis.

Preferably the growth hormone fragment has the ability to stimulate the activity of hormone-sensitive lipase, a key enzyme in lipolysis, and to inhibit acetyl CoA carboxylase, a key enzyme in lipogenesis.

Preferably the growth hormone fragment comprises at least the disulphide-bonded loop of a mammalian growth hormone.

The term “lipid metabolic growth hormone fragment” also encompasses peptides which are analogues of the native carboxy-terminal sequences of mammalian growth hormones, provided that the analogue retains one or more of the biological activities referred to above. Such analogues may be derived from natural sources, produced by recombinant DNA technology, or synthesised using conventional peptide synthetic methods. Such peptides synthetic methods are to be understood to include combinatorial methods. Preferably such analogues include a disulphide bond which confers a cyclic configuration on the peptide. In particular, all of the peptides disclosed in PCT/AU98/00724 are to be understood to be within the scope of this invention.

Preferably the growth hormone fragment is amino acids 182-189 (hGH 182-189), more preferably amino acid 177-191 of human growth hormone (hGH 177-191), even more preferably Tyr-hGH 177-191 (AOD9604). However, it will be clearly understood that the invention is also applicable to growth hormone fragments derived from growth hormones of other mammalian species, including but not limited to those of domestic mammals such as cattle, sheep, pigs and horses, companion animals such as cats and dogs, and zoo animals including felids, canids, and non-human primates. There is strong conservation of the sequence of this region of growth hormone across species, as set out in PCT/AU98/00724 and references cited therein.

It will also be appreciated that any non-peptide compound which is found to increase the expression of the beta 3 adrenergic receptor by the same mechanism as hGH and AOD 9604 is within the scope of this invention, including non-peptide analogues of growth hormone fragments.

The beta 3 agonists of the invention encompasses any agonist of the beta 3 receptor, preferably agonists selective for the human beta 3 receptor particularly useful in treating human obesity.

### BRIEF DESCRIPTION OF THE FIGURES

FIG. 1 shows the results of comparative experiments in vitro on lipolysis using AOD 9604, and the beta 3 agonist BRL 37344, either alone or in combination.

FIG. 2 shows clearly the synergistic effect on lipolysis obtained by a 4 hour in vitro incubation of hGH (FIG. 2A) or AOD 9604 (FIG. 2B) in combination with a sub-maximal dose of BRL 37344 for a further hour.

FIG. 3 shows the increase in expression of beta 3 messenger RNA after a 4 hour incubation with AOD 9604.

FIG. 4 shows the increase in expression of beta 3 messenger RNA following a 2 week chronic administration of hGH or AOD 9604 obese (ob/ob) mice in white adipose tissue (a) or brown adipose tissue (b).

### DETAILED DESCRIPTION OF THE INVENTION

The methods and compositions of the invention are useful for reducing or controlling obesity.

The term “mammal” as used herein refers to any mammalian animal which suffers from or is prone to obesity. Mammals for the purposes of the invention include, but are not limited to bovine, canine, equine, feline, porcine and preferably humans. It will be appreciated by those skilled in the art that physiologically all mammals are very similar. There is strong conservation of the growth hormone amino acid sequence across all mammalian species and there are strong indications that the methods and compositions of the invention would be useful for reducing or controlling obesity in all mammals.

The methods of this invention involve in one embodiment, (1) the administration of an agent which increases the expression of beta-3 adrenergic receptors in the mammal, prior to, together with, or subsequent to the administration of an agonist of the beta 3 adrenergic receptor of the mammal; or (2) the administration of a combination of an agent which increases the expression of beta-3 adrenergic receptors in the mammal and an agonist of the beta 3 adrenergic receptor.

As used herein, the term “therapeutically effective amount” is meant an amount of a composition of the present invention effective to yield a desired therapeutic response. For example to prevent or treat obesity in a mammal.

The specific “therapeutically effective amount” will, obviously, vary with such factors as the physical condition of the mammal, the type of mammal being treated, the duration of the treatment, the nature of concurrent therapy (if any), and the specific formulations employed and the structure of the compositions or their derivatives.

As used herein, a “pharmaceutical carrier” is a pharmaceutically acceptable solvent, suspending agent or vehicle for delivering the agent of the invention to the mammal. The carrier may be liquid or solid and is selected with the planned manner of administration in mind.

As used herein, the term “cell” includes but is not limited to mammalian cells (eg., mouse cells, rat cells or human cells).

The agent which increases the expression of beta-3 adrenergic receptors in the mammal and the agonist of the beta 3 adrenergic receptor may be administered orally, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, intrasternal injection or infusion techniques.

The present invention also provides suitable topical, oral, and parenteral pharmaceutical formulations for use in the novel methods of treatment of the present invention. The compounds of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable-preparations. The tablets contain the active ingredient in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U. S. Pat. Nos. 4,256,108; 4,160,452; and 4,265,874 to form osmotic therapeutic tablets for control release.

The agent which increases the expression of beta-3 adrenergic receptors in the mammal or agonist of the beta 3 adrenergic receptor useful in the method of the invention can be administered, for in vivo application, parenterally by injection or by gradual perfusion over time independently or together. Administration may be orally, intravenously, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents-are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

Generally, the terms “treating”, “treatment” and the like are used herein to mean affecting a subject, tissue or cell to obtain a desired pharmacologic and/or physiologic effect. The effect may be prophylactic in terms of completely or partially preventing obesity or sign or symptom thereof, and/or may be therapeutic in terms of a partial or complete cure of obesity. “Treating” as used herein covers any treatment of, or prevention of obesity in a mammal, particularly a human, and includes: (a) preventing the obesity from occurring in a mammal that may be predisposed to obesity, but has not yet been diagnosed as having it; (b) inhibiting the obesity, ie., arresting its development; or (c) relieving or ameliorating obesity, ie., cause regression of obesity.

The invention includes various pharmaceutical compositions useful for ameliorating obesity. The pharmaceutical compositions according to one embodiment of the invention are prepared by bringing growth hormone fragment and beta 3 agonist BRL 37344 into a form suitable for administration to a mammal using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412,1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories. For treatment of a mammal, depending on activity of the compound, manner of administration, nature and severity of the obesity and age of the mammal, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the obesity and general state of the mammal. Typically, dosages used in vitro may provide useful guidance in the amounts useful for in situ administration of the pharmaceutical composition, and animal models may be used to determine effective dosages for treatment of obesity. Various considerations are described, eg., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleagenous suspension. This suspension may be formulated according to known methods using those suitable dispersing or wetting agents and suspending agents which have been mentioned above. The sterile injectable preparation may also a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Growth hormone, fragments of growth hormone or the beta 3 agonist may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

It will be understood that the specific dose level for any particular mammal will depend upon a variety of factors including the activity of the specific compound employed, the age, body weight, general health, sex, diet, time of administration, route of administration, rate of excretion, drug combination and the severity of the obesity undergoing therapy. Appropriate dose levels for growth hormone in humans range from 1 microgram per kg to 30 micrograms per kg. Appropriate dose levels for the growth hormone peptides in rats and mice are from 50 micrograms per kg to 2000 micrograms per kg, given IV or orally. Appropriate dose levels of the beta-3 agonist will vary depending on the compound.

In addition, some of the compounds of the instant invention may form solvates with water or common organic solvents. Such solvates are encompassed within the scope of the invention.

The compounds of the present invention may additionally be combined with other compounds to provide an operative combination. It is intended to include any chemically compatible combination of chemotherapeutic agents, as long as the combination does not eliminate the activity of growth hormone, the growth hormone fragment or beta 3 agonist of this invention.

The invention will now be further described by way of reference only to the following figures. It should be understood, however, that the figures following are illustrative only, and should not be taken in any way as a restriction on the generality of the invention described above. In particular, while the invention is described in detail in relation to human growth hormone, the human growth hormone fragment AOD 9604, used either alone or in combination with the beta 3 agonist BRL37344, it will be clearly understood that the findings herein are not limited to AOD 9604 or BRL37344. For example, analogues, derivatives or homologues thereof of these molecules may be used for treatment of the obesity.

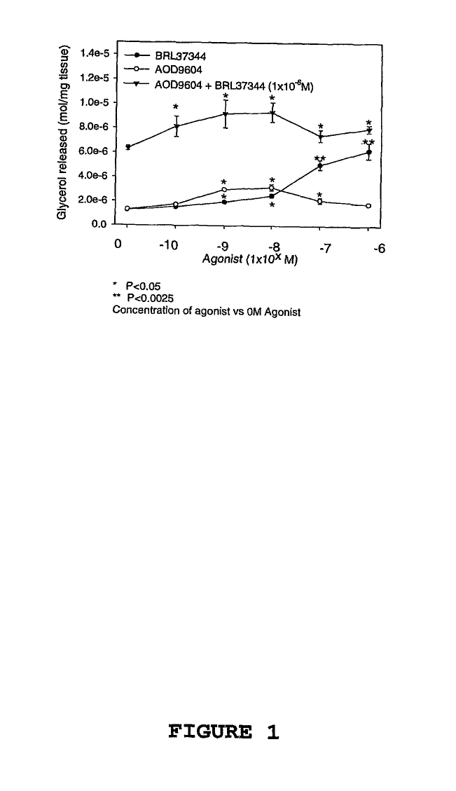

Referring now to FIG. 1, 3T3-F442 cells (an immortal mouse adipocyte cell line) were incubated for 1 hour with varying concentrations of the rodent beta 3 agonist BRL 37344. A sigmoid-shaped dose response was obtained, with a maximal effect at 1 μM concentration of approximately 6 μM per milligram protein of glycerol released. Next, another sample of the cells were incubated with varying concentrations of AOD9604 for 1 hour, showing a bell-shaped acute lipolytic response, typical of the acute in vitro properties of the growth hormone fragments. AOD9604 is not an agonist at the beta 3 adrenergic receptor. In a third sample, AOD 9604 and BRL 37344 were co-administered with varying concentrations of AOD 9604, together with the maximal concentration of 1 μM of the BRL 37344. A clear increase in effect above and beyond the maximal effect obtained with the BRL 37344 was shown.

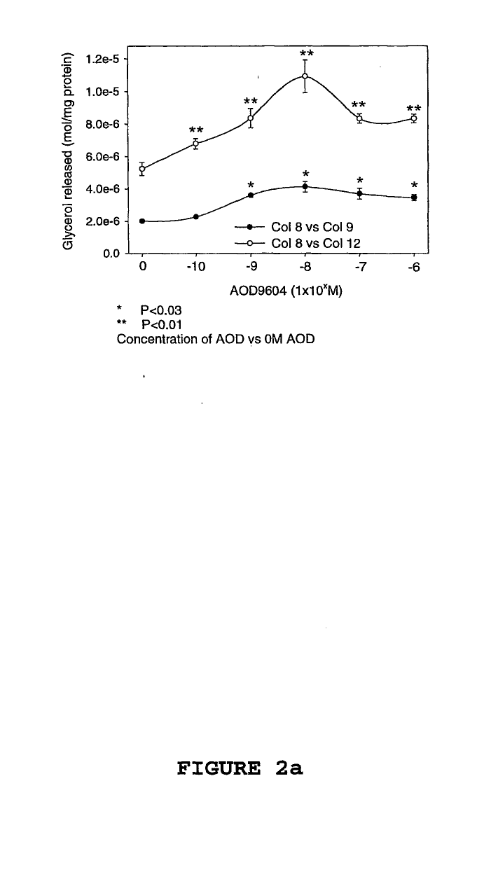

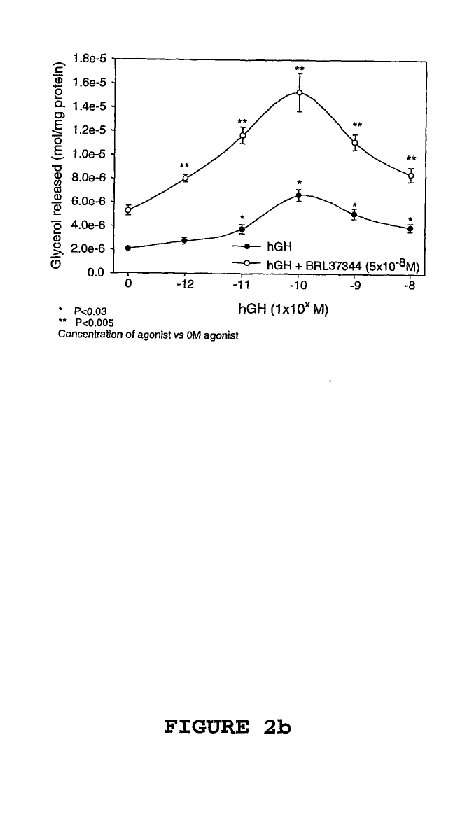

Referring now to FIG. 2, an experiment was performed to determine whether hGH (FIG. 2A) or AOD 9604 (FIG. 2B) sensitises the adipocytes to the actions of the beta 3 agonists. First, the adipocytes were incubated with hGH or AOD 9604 at varying concentrations for four hours. The cells were then further incubated with a half-maximal concentration of BRL 37344 of 50 nanomolar for a further hour. The results show that the lipolytic effect of the beta 3 agonist is enhanced by the 4-hour pre-incubation with either hGH or AOD 9604.

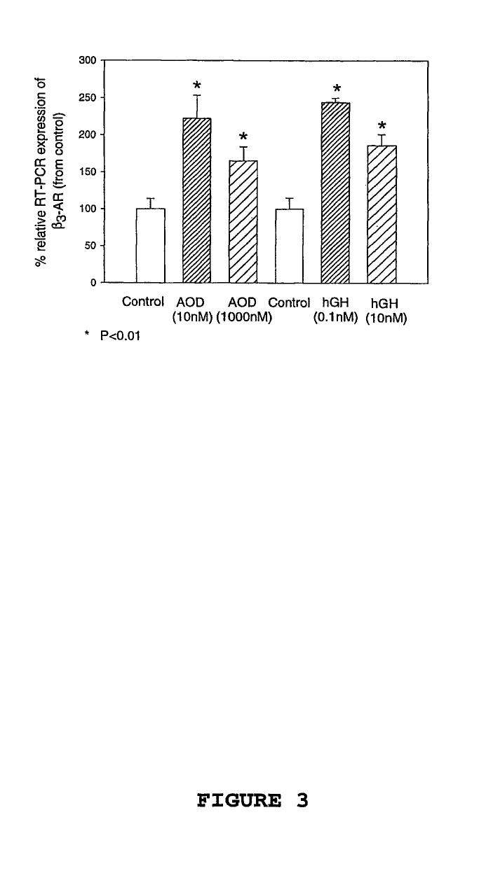

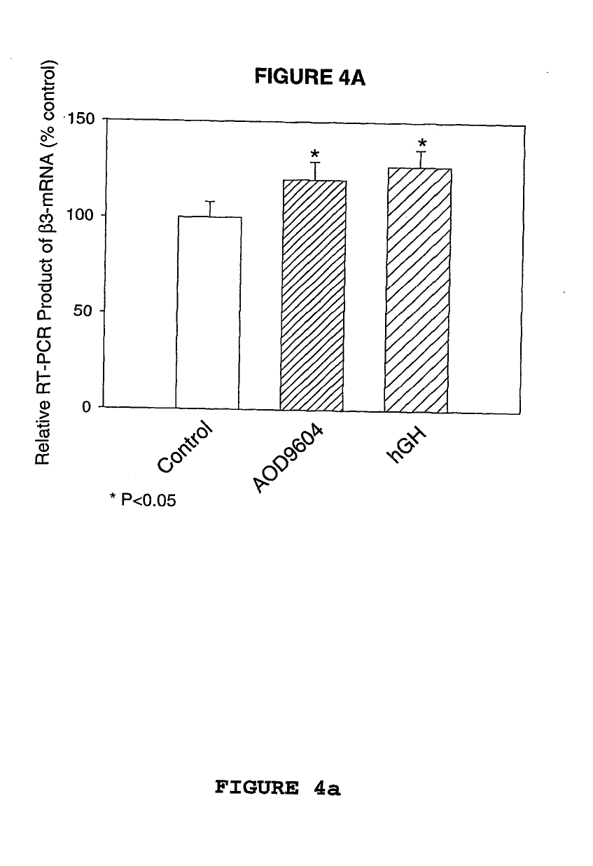

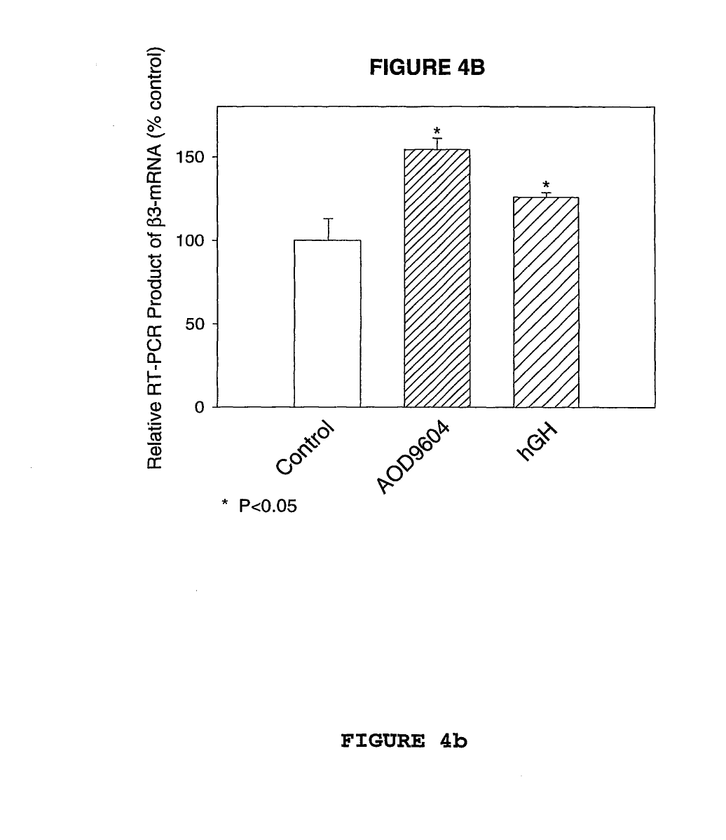

Referring now to FIG. 3, the effect of the four-hour incubation of the adipocytes with hGH (FIG. 3A) or AOD 9604 (FIG. 3B) at two concentrations on expression of beta 3 messenger RNA was assessed using PCR/RT-PCR analysis of the content of beta 3 messenger RNA, using β-actin to normalise the data.

Chronic treatment of ob/ob mice with effective weight reducing doses of hGH (1.0 mg/kg/day) or AOD9604 (0.25 mg/kg/day) for 14 days also showed a similar increase of beta 3 messenger RNA in white (A) and brown (B) adipocytes of the treated animals compared with control ob/ob mice.

Thus, chronic treatment using AOD9604 not only produces a significant reduction in fat mass of the treated animals, but sensitises the adipocytes to the lipolytic actions of beta 3 agonists by enhancing beta 3 receptor number. It is believed that the obesity reducing effect of AOD9604 is at least in part a result of this sensitisation to the lipolytic affects of endogenous catecholamines (ie. epinephrine). Accordingly, chronic co-administration of AOD9604 and a beta 3 agonist will enhance the anti-obesity action of either agent administered alone.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The claims defining the invention are as follows:

1. A method of treating obesity in a mammal, comprising the step of administering to the mammal in need thereof a therapeuticallyeffective amount of an agent which increases the expression of beta-3 adrenergic receptors in the mammal, together with an agonist of the beta 3 adrenergic receptor of the mammal.

2. A method according to claim 1, wherein the agent is growth hormone or a lipid metabolic growth hormone fragment, or a non-peptide analogue thereof.

3. A method according to claim 1, wherein the agonist of the beta 3 adrenergic receptor of the mammal is a selective beta 3 agonist to the human beta 3 receptor.

4. A method according to claim 1, wherein the mammal is a human.

5. A method according to claim 1, wherein the mammal is a pig.

6. A method according to claim 1, wherein the beta 3 agonist is either a selective or non-selective agonist to the beta 3 adrenergic receptor.

7. A method according to claim 1, wherein the beta 3 agonist is ractopamine.

8. A method according to claim 2, wherein the lipid metabolic growth hormone fragment is a polypeptide fragment from the carboxy-terminal region of the amino acid sequence of a mammalian growth hormone.

9. A method according to claim 8, wherein the growth hormone fragment comprises at least the disulphide-bonded loop of a mammalian growth hormone.

10. A method according to claim 8, wherein the growth hormone fragment comprises amino acids 182-189 from the human growth hormone molecule.

11. A method according to claim 8, wherein the growth hormone fragment comprises amino acids 177-191 of human growth hormone molecule.

12. A method according to claim 8, wherein the growth hormone fragment comprises amino acids Tyr-hGH 177-191 (AOD9604).

13. A method of treating obesity in a mammal, comprising the step of administering to the mammal in need thereof a therapeutically-effective amount of a non-peptide compound which increases the expression of beta 3 adrenergic receptor in the mammal, together with an agonist of the beta 3 adrenergic receptor.

14. A method according to claim 13, wherein the non-peptide compound is an analogue of growth hormone.

15. A method according to claim 13 or claim 14, wherein the beta 3 agonist is ractopamine.
